(19) United States
(12) Patent Application Publication
(10) Pub. No.: US 2014/0371851 A1
(43) Pub. Date: Dec. 18, 2014

(54) HAPTICS FOR INTRAOCULAR DEVICES

(71) Applicant: Eli Aharoni, Tel Aviv (IL)

(72) Inventor: Eli Aharoni, Tel Aviv (IL)

(21) Appl. No.: 13/918,969

(22) Filed: Jun. 16, 2013

(51) Int. Cl. A61F 2/16

(52) U.S. Cl. CPC A61F 2/1613
USPC 623/6.51

(57) ABSTRACT

An intraocular device having anterior and posterior faces, haptics extending from the intraocular device, each of the haptics including a slender loop element with a proximal end attached to the intraocular device, the slender loop element having a length, width and thickness, the length following a curved path and the width being generally parallel to a central anterior-posterior axis of the intraocular device, and a distal hook portion extending from a distal end of the slender loop element, the distal hook portion including an anterior edge tilted towards the central anterior-posterior axis of the intraocular device.

### FIELD OF THE INVENTION

The present invention relates generally to intraocular devices, such as intraocular lenses (IOLs) and particularly to haptics for intraocular devices, such as an IOL.

### BACKGROUND OF THE INVENTION

Haptics are clearly defined in the art as the interface elements of an intraocular device that touch the eye structure (“haptic” is from the Greek word for touch). Haptics are typically outwardly extending (e.g., radially, tangentially, or the like) structures that are attached to the optic and support the optic within the eye by pressing against adjacent tissue.

Both resilient and rigid haptics are known. Resilient haptics are generally resilient wires made of plastics or any other biologically inert material, but which are sufficiently stiff so that when a compressive force is applied thereto, they distort but do not buckle or collapse. Rigid haptics do not deform significantly under compressive or tensile forces; they may be made of significantly stiffer materials than resilient haptics or may be more rigid due to their shape and construction, such as plate haptics.

Although haptics are mostly used in the capsular bag, haptics are also used to affix the device to ocular structure outside the bag. For example, haptics have been inserted in sclerotomies, which are incisions made in the sclera. Currently known fixation methods include bonding the haptics to the ocular structure with an adhesive; making a flap (rectangular, square, triangular, Y-shape or other shapes), inserting the haptic and covering it with the flap, and suturing; or inserting and fixing a haptic in a scleral tunnel. However, these methods have disadvantages. For example, it is difficult to insert haptic loops into sclerotomies. Tilting and decentration may occur due to poor fixation. The haptic loops may become distorted due to manipulation by tools, such as intraocular forceps.

### SUMMARY OF THE INVENTION

The present invention seeks to provide improved haptics for intraocular devices, as is described further in detail hereinbelow.

There is thus provided in accordance with an embodiment of the present invention an assembly including an intraocular device having anterior and posterior faces, haptics extending from the intraocular device, each of the haptics including a slender loop element with a proximal end attached to the intraocular device, the slender loop element having a length, width and thickness, the length following a curved path and the width being generally parallel to a central anterior-posterior axis of the intraocular device, and a distal hook portion extending from a distal end of the slender loop element, the distal hook portion including an anterior edge tilted towards the central anterior-posterior axis of the intraocular device.

Non-limiting features of the invention include the following, among others:

The distal hook portion includes a posterior edge tilted away from the central anterior-posterior axis of the intraocular device.

The distal hook portion is round at its distal tip and tapers to be thinner from where it extends from the slender loop element.

The distal hook portion is thinner than the slender loop element.

The thickness of the slender loop elements is less than the width.

The distal hook portion is formed with a through hole.

The proximal end of the slender loop element is generally tangential to an outer periphery of the intraocular device.

The distal hook portion and the slender loop element can have different rigidity; the slender loop element and the intraocular device can have different rigidity.

The anterior-most surface of the distal hook portion is not posterior to the anterior-most surface of the intraocular device; the anterior-most surface of the slender loop element is not posterior to the anterior-most surface of the intraocular device.

In accordance with an embodiment of the present invention the intraocular device includes an IOL, a scleral fixation bag or an implantable miniature telescope.

In accordance with an embodiment of the present invention, the flat and angled loop edges of the haptics help fixate and stabilize the lens or other ocular device. The flat loop profile is shaped for tangential placement in the sclera (i.e., tangent to the sclera), providing better stabilization and preventing tilting and decentration of the lens or other intraocular device. The distal hook portion eases the manipulation of the assembly/device in the eye. The hooked loops at the distal haptic ends significantly facilitate implantation directly into sclerotomies.

### BRIEF DESCRIPTION OF THE DRAWINGS

The present invention will be understood and appreciated more fully from the following detailed description taken in conjunction with the appended drawings in which:

FIG. 1 is a simplified pictorial illustration of an intraocular assembly (IOL) with haptics, constructed and operative in accordance with an embodiment of the present invention;

FIG. 2A is a simplified pictorial illustration of the intraocular assembly of FIG. 1 implanted in an eye, viewed along the anterior-posterior axis, wherein the haptics are fixed in a curved sclerotomy, in accordance with an embodiment of the present invention;

FIG. 2B is a simplified illustration of the haptics in the sclerotomy, viewed transverse to the anterior-posterior axis;

FIG. 3 is a simplified pictorial illustration of an intraocular assembly (implantable miniature telescope) with haptics, constructed and operative in accordance with an embodiment of the present invention; and

FIG. 4 is a simplified pictorial illustration of an IOL with haptics, constructed and operative in accordance with another embodiment of the present invention.

### DETAILED DESCRIPTION OF THE PRESENT INVENTION

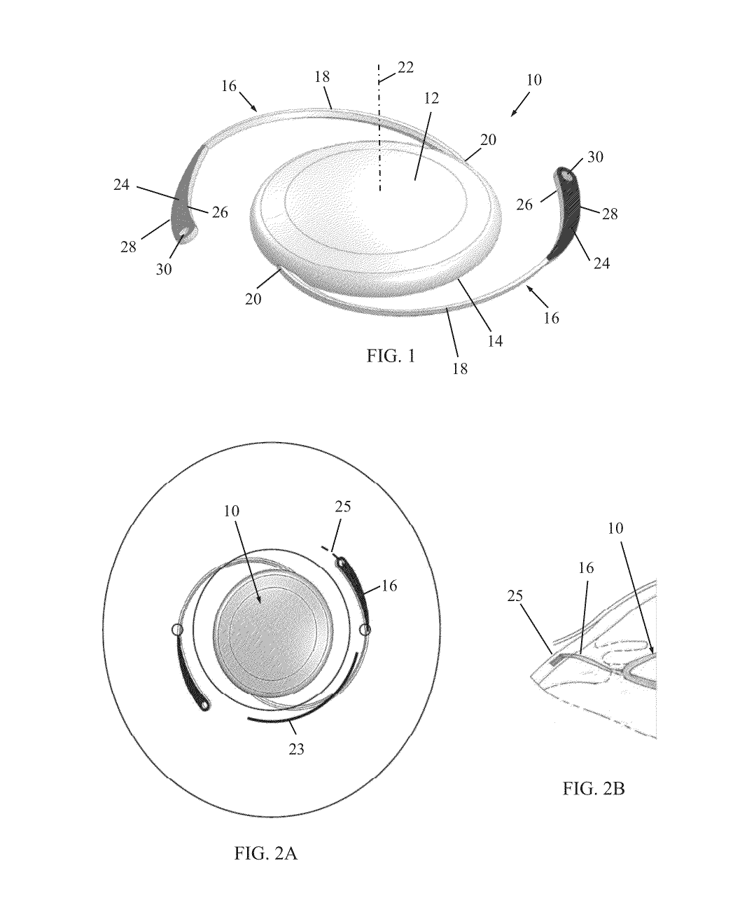

Reference is now made to FIGS. 1-2B, which illustrate an intraocular assembly, constructed and operative in accordance with an embodiment of the present invention.

The assembly includes an intraocular device 10 having anterior and posterior faces 12 and 14. In this embodiment, intraocular device 10 is an IOL (such as, but not limited to, a monofocal IOL, multifocal IOL, accommodating IOL and others). Haptics 16 extend from intraocular device 10. Each haptic 16 includes a slender loop element 18 with a proximal end 20 attached to intraocular device 10. Slender loop element 18 has a length, width and thickness; its length follows a curved path and its width is generally parallel to a central anterior-posterior axis 22 of intraocular device 10. The outer diameter of intraocular device 10 with haptics 16 is, without limitation, 15 mm for proper mounting in the sclera.

A distal hook portion 24 extends from a distal end of slender loop element 18. Distal hook portion 24 includes an anterior edge 26 tilted towards the central anterior-posterior axis 22 of intraocular device 10. Preferably, but not necessarily, the posterior edge 28 of distal hook portion 24 is tilted away from central anterior-posterior axis 22. Preferably, but not necessarily, distal hook portion 24 is flat. The shape of distal hook portion 24 may match the shape of its insertion place in the sclera. Distal hook portion 24 is rounded at its distal tip and tapers to be thinner from where it extends from slender loop element 18. Distal hook portion 24 may be formed with a through hole 30, for grasping or dialing or other adjustments. Through hole 30 is preferably central and not shifted to one side. In one embodiment, distal hook portion 24 is thinner than slender loop element 18. In one embodiment, the thickness of slender loop elements 18 is less than its width; in other embodiments they are equal.

The proximal end of slender loop element 18 is generally tangential to the outer periphery of intraocular device 10.

The lens portion or optic, as well as the other portions of the assembly including the haptics, may be made from one or more materials that are biocompatible and optically transparent, and can be hydrophilic or hydrophobic. The material may be rigid or flexible, hard or soft, such as without limitation, methacrylates (e.g., polymethyl methacrylate), olefins (e.g., polypropylene), and silicones.

The distal hook portion 24 and the slender loop element 18 can have different rigidity. Similarly, slender loop element 18 and intraocular device 10 can have different rigidity.

Reference is now made to FIGS. 2A and 2B. The intraocular device 10 is implanted, such as through an incision 23 in the limbus. The haptics 16 are fixed in curved sclerotomies 25 and properly center the device 10.

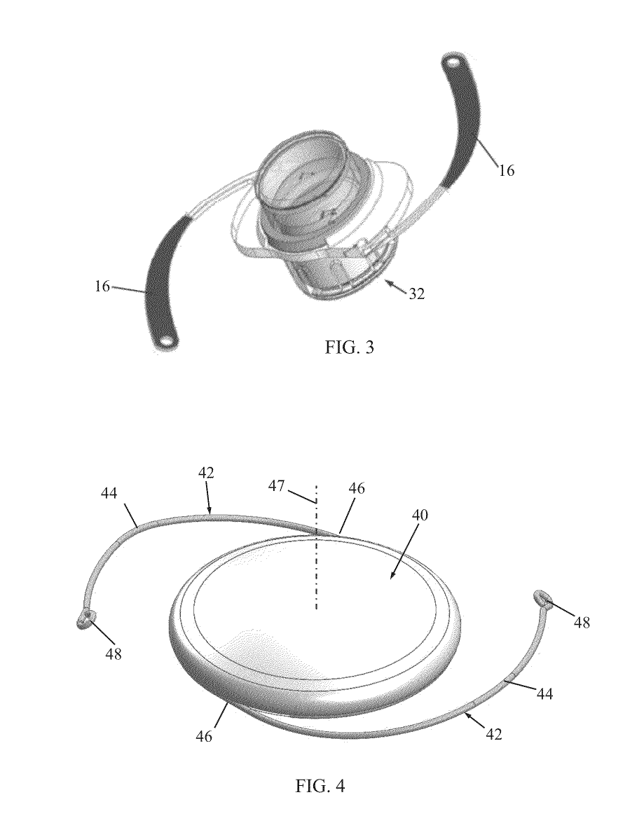

Reference is now made to FIG. 3, in which the intraocular assembly is an implantable miniature telescope 32 with haptics 16, constructed and operative in accordance with an embodiment of the present invention. The haptics 16 are as described above. The haptics can be used with other intraocular devices, such as a scleral fixation bag, which is described in a copending application.

Reference is now made to FIG. 4, which illustrates an IOL 40 with haptics 42, constructed and operative in accordance with another embodiment of the present invention. As in the other embodiments of the invention, each haptic 42 includes a slender loop element 44 with a proximal end 46 attached to IOL 40 and a distal hook portion 48 whose anterior edge is tilted towards the central anterior-posterior axis 47 of IOL 40. In this embodiment, slender loop element 44 is a slender wire and distal hook portion 48 is a rounded eyelet bent or otherwise formed from the end of the slender wire.

What is claimed is:

1. An assembly comprising:
an intraocular device having anterior and posterior faces;
haptics extending from said intraocular device, each of said haptics comprising a slender loop element with a proximal end attached to said intraocular device, said slender loop element having a length, width and thickness, the length following a curved path and the width being generally parallel to a central anterior-posterior axis of said intraocular device; and
a distal hook portion extending from a distal end of said slender loop element, said distal hook portion comprising an anterior edge tilted towards the central anterior-posterior axis of said intraocular device.

2. The assembly according to claim 1, wherein said distal hook portion comprises a posterior edge tilted away from the central anterior-posterior axis of said intraocular device.

3. The assembly according to claim 1, wherein said distal hook portion is rounded at its distal tip and tapers to be thinner from where it extends from said slender loop element.

4. The assembly according to claim 1, wherein said distal hook portion is flat.

5. The assembly according to claim 1, wherein said distal hook portion is thinner than said slender loop element.

6. The assembly according to claim 1, wherein the thickness of said slender loop elements is less than the width.

7. The assembly according to claim 1, wherein said distal hook portion is formed with a central through hole.

8. The assembly according to claim 1, wherein the proximal end of said slender loop element is generally tangential to an outer periphery of said intraocular device.

9. The assembly according to claim 1, wherein said distal hook portion and said slender loop element have different rigidity.

10. The assembly according to claim 1, wherein said slender loop element and said intraocular device have different rigidity.

11. The assembly according to claim 1, wherein an anterior-most surface of said distal hook portion is not posterior to an anterior-most surface of said intraocular device.

12. The assembly according to claim 1, wherein an anterior-most surface of said slender loop element is not posterior to an anterior-most surface of said intraocular device.

13. The assembly according to claim 1, wherein said slender loop element is a slender wire and said distal hook portion is a rounded eyelet formed from an end of said slender wire.

14. The assembly according to claim 1, wherein said intraocular device comprises an IOL.

15. The assembly according to claim 1, wherein said intraocular device comprises an implantable miniature telescope.
